(19) United States
(12) Patent Application Publication
(10) Pub. No.: US 2010/0322864 A1
(43) Pub. Date: Dec. 23, 2010

(54) TELEMETRY THROUGH REMOTE DETECTION OF NMR-ACTIVE PARTICLES

(75) Inventors: Charles M. Marcus, Cambridge, MA (US); Jonathan Marmurek, Cambridge (MA); Jacob W. Aptekar, Denver, CO (US); Geoffrey Von Maltzahn, Cambridge, MA (US)

Correspondence Address: CHOATE, HALL & STEWART LLP, TWO INTERNATIONAL PLACE, BOSTON, MA, 02110, US

(73) Assignees: PRESIDENT AND FELLOWS OF HARVARD COLLEGE, Cambridge, MA (US); MASSACHUSETTS INSTITUTE OF TECHNOLOGY, Cambridge, MA (US)

(21) Appl. No.: 12/812,069

(22) PCT Filed: Jan. 9, 2009
(86) PCT No.: PCT/US09/30672

§ 371 Date: Aug. 27, 2010

Related U.S. Application Data
(60) Provisional application No. 61/020,248, filed on Jan. 10, 2008.

(51) Int. Cl. A61K 49/06; G01R 33/44

(52) U.S. Cl. 424/9.3; 324/309

(57) ABSTRACT

Various methods of telemetry for nuclear magnetic resonance applications are described. NMR-active particles are introduced into a system which is to undergo an NMR measurement. In various embodiments, the NMR-active particles have a resonance peak in a spectral region which is substantially free from any NMR signal originating from material native to the system. In some embodiments, the NMR-active particles are chemically functionalized to target a constituent within the system. In certain applications, changes in the detected resonance peak can be used to quantify certain characteristics about the system, e.g., a concentration of an analyte, whether a targeted constituent is present within the system.

### CROSS-REFERENCE TO RELATED U.S. APPLICATIONS

The present application claims priority to U.S. provisional Patent Application No. 61/020,248 filed on 10 Jan. 2008, which is incorporated herein by reference.

### GOVERNMENT FUNDING

The work described herein was conducted within a research program supported in part with U.S. government funding under R01CA124427-02, U54 CA119335, and 5U54CA119349-03 awarded by the National Institutes of Health, and DMR-0213805 awarded by the National Science Foundation. The U.S. Government has certain rights in these inventions.

### BACKGROUND

Nuclear magnetic resonance (NMR) is a physical phenomenon associated with the spin angular momentum of atomic nuclei, and is currently utilized for a variety of medical and scientific diagnostic measurements. Magnetic resonance imaging (MRI), a technique based on NMR, has become a powerful non-invasive diagnostic technique for viewing the internal structures of organisms and materials. Magnetic resonance spectroscopy is another NMR-based technique which can provide details about the structure and/or composition of geological samples, cells, proteins and complex molecular structures for the fields of geology, biology, biochemistry and organic chemistry.

Various types of remote-detection measurements based on NMR have found applications in the areas of spectroscopic and imaging analyses of heterogeneous mixtures, chemical analysis, geological exploration and magnetic resonance spectroscopy. However, the detected NMR signals in these applications are typically of low quality and require long data acquisition times. Further, conventional imaging techniques base on NMR measurements provide low spatial resolution. For example, the length of time required to acquire a single scan is often tens of minutes, and voxel dimensions for magnetic resonance images are routinely larger than 10 milliliters.

In some approaches, superparamagnetic particles have been used in concert with MRI to perform in vivo telemetry in agglomeration assays, where the coherence time, or spin-spin relaxation time, T₂ of the proton signal originating from water molecules is strongly dependent on the agglomeration of the superparamagnetic particles. The superparamagnetic particles in the vicinity of the water molecules affect and alter their T₂ signal by affecting the local magnetic field. For these measurements, semi-permeable micro-compartments filled with a mixture of water and the superparamagnetic particles are implanted into a subject. This requires spatially-selective magnetic resonance excitations to measure T₂ relaxation rates over the confined volumes, is time-inefficient, and can be difficult to implement. Additionally, a high level of control over the magnetic field profile is required.

A further difficulty exists with spectroscopic and agglomeration NMR techniques. Since both measurements detect a proton signal from a native atomic species, their sensitivity suffers from a substantial NMR background signal originating from the examined region itself. This background signal degrades the quality of the recorded data.

### SUMMARY

The inventive embodiments disclosed herein include methods of telemetry for nuclear magnetic resonance which are useful for determining remotely whether a system exhibits a particular characteristic. In various embodiments, NMR-active particles are introduced into a system which is to undergo an NMR measurement. The system is subjected to NMR excitation and nuclear magnetic resonance signals derived from the NMR-active particles are detected using NMR apparatus and analyzed. Analysis of the detected signals can determine whether the system exhibits or does not exhibit a particular characteristic.

In various aspects, a method of telemetry for nuclear magnetic resonance comprises providing NMR-active particles having an NMR resonance peak in a spectral region which is substantially free from any NMR signal originating from a system of which an NMR measurement will be made. The NMR-active particles can be small in size, e.g., sub-millimeter, sub-micron, nanometer scale, and act as imaging agents. The method of telemetry can further comprise introducing the NMR-active particles into the system, and detecting a shift in the resonance peak of the NMR-active particles. In some embodiments, the method further comprises enhancing a nuclear magnetic resonance signal originating from the NMR-active particles by dynamic nuclear polarization, where the dynamic nuclear polarization is performed in situ or ex situ. In yet additional embodiments, the method of telemetry further comprises associating a concentration with the detected shift in resonance peak.

The inventive embodiments also include a method of telemetry for nuclear magnetic resonance assays. The method can comprise steps of providing NMR-active particles having an NMR resonance peak in a spectral region which is substantially free from any NMR signal originating from other components in an assay system; introducing the NMR-active particles into the assay system; introducing an analyte into the assay system; and detecting a shift in the resonance peak of the NMR-active particles. The method of telemetry for assays can further comprise associating a concentration of the analyte with the detected shift in resonance peak. In some embodiments, the method further comprises enhancing a nuclear magnetic resonance signal originating from the NMR-active particles by dynamic nuclear polarization, where the dynamic nuclear polarization is performed in situ or ex situ.

In certain embodiments, a method of telemetry for nuclear magnetic resonance comprises providing NMR-active particles having an NMR resonance peak in a spectral region which is substantially free from any NMR signal originating from other components within a system; introducing the NMR-active particles into the system; and detecting or measuring one or more characteristics or aspects of signals, and/or their changes, provided by the NMR-active particles. An embodiment of this method of telemetry can further comprise forming an image based upon data from the one or more detected aspects and/or their changes. An embodiment of this method of telemetry can further comprise weighting the formed image by, or associating the formed with, data from one or more different detected aspects and/or their changes, e.g., an image formed from signal intensity weighted by data representing a frequency change in the NMR-particles' resonant frequency.

In certain embodiments the NMR-active particles are chemically functionalized. In some embodiments, the NMR-active particles have undergone isotopic enrichment or isotopic depletion. In various aspects, the resonance peak of the NMR-active particles has a signal strength greater than about 2 times the background NMR signal level, greater than about 5 times the background NMR signal level, greater than about 10 times the background NMR signal level, and yet in some embodiments greater than about 20 times the background NMR signal level.

In certain embodiments, the methods of telemetry are carried out using spatially resolving measurement techniques. For example, magnetic field gradients may be used so that the detection of the shift in resonance peak or a change in resonance peak intensity is done using spatially resolving measurement techniques. In various aspects, the spatial resolution is between about 5 milliliters and about 10 milliliters, between about 2.5 milliliters and about 5 milliliters, and in some cases between about 1 milliliter and about 2.5 milliliters. In some embodiments, the methods of telemetry are carried out without using spatially resolving measurement techniques.

In various embodiments, an NMR measurement to detect the shift in resonance peak requires between about 10 minutes and about 20 minutes, between about 5 minutes and about 10 minutes, between about 2.5 minutes and about 5 minutes, and yet in some embodiments between about 1 minute and about 2.5 minutes.

The foregoing and other aspects, embodiments, and features of the present teachings can be more fully understood from the following description in conjunction with the accompanying drawings. All literature and similar material cited in this application, including, but not limited to, patents, patent applications, articles, books, treatises, and web pages, regardless of the format of such literature and similar materials, are expressly incorporated by reference in their entirety.

### BRIEF DESCRIPTION OF THE DRAWINGS

The skilled artisan will understand that the figures, described herein, are for illustration purposes only. It is to be understood that in some instances various aspects of the invention may be shown exaggerated or enlarged to facilitate an understanding of the invention. In the drawings, like reference characters generally refer to like features, functionally similar and/or structurally similar elements throughout the various figures. The drawings are not necessarily to scale, emphasis instead being placed upon illustrating the principles of the teachings. The drawings are not intended to limit the scope of the present teachings in any way.

FIG. 1 represents the dynamics of motion of a nuclear magnetic moment 110 in a substantially uniform and static magnetic field \vec{B}. The magnetic moment will precess, tracing out path 120, and execute gyroscopic motion.

FIG. 2A represents a collection of atoms or molecules 210 for which the magnetic moments 110 are randomly oriented.

FIG. 2B represents a collection of atoms or molecules that have been polarized by a magnetic field. A fraction of the atoms 220 have their magnetic moments oriented in a preferred direction.

FIG. 3 is a graphical representation of the NMR spectra for an NMR-active particle and a system into which the particle may be introduced. In some embodiments, the system's spectrum 301 exhibits substantially no resonant peaks or signal in the vicinity of the NMR-active particle's spectral peak 350.

FIG. 4A depicts a particle 410 with a functionalized surface and a targeted constituent 450. Targeting ligands 420 on the surface of the particle bind to receptors 460 located on a targeted constituent.

FIG. 4B illustrates a bound NMR-active-particle/targeted-constituent pair.

FIGS. 4C-4D depict functionalized NMR-active particles which include an encapsulating shell 480.

FIG. 5A is a graphical representation of a spectral peak of an NMR-active particle. For example, the resonance peak 510 may correspond to the NMR signal strength in the vicinity of magnetic resonance excitation frequency ωₚ for an unbound particle 410, e.g., as depicted in FIG. 4A.

FIG. 5B is a graphical representation depicting changes in the NMR spectral characteristics of FIG. 5A that can occur when a particle binds with a targeted constituent, e.g., as depicted in FIG. 4B.

FIG. 6 depicts an agglomeration of targeted constituents 650 bound to two types of particles. An NMR-active particle 410 provides an NMR signal when excited, and a paramagnetic or superparamagnetic particle 610 can alter the NMR signal when bound in close proximity to the NMR-active particle.

FIGS. 7A-7B depict embodiments of methods for NMR telemetry using NMR-active particles.

FIG. 8 shows several plots of normalized NMR signal amplitude versus frequency for NMR-active particles of different average sizes. The data has been shifted to zero frequency.

The features and advantages of the present invention will become more apparent from the detailed description set forth below when taken in conjunction with the drawings.

### DETAILED DESCRIPTION

By way of overview, the inventive methods of telemetry for nuclear magnetic resonance utilize NMR-active particles which can be introduced into a system. At least some atoms within the particles have non-zero nuclear spin. These NMR-active particles can be incorporated directly into a system to provide an NMR signal when probed by an applied excitation field. The resulting NMR signal can be detected by electronic instrumentation and be diagnostic of the condition, structure or composition of the system.

In certain embodiments, the NMR-active particles are chemically functionalized. As an example, the surfaces of the particles can be functionalized so as to induce attachment of the particles to a targeted constituent within a system.

As used herein, the term “particles” encompasses small particles of NMR-active material. The size of the particles can be sub-millimeter, sub-micron, and yet nanometer scale. As used herein, the term “system” encompasses a sample, specimen, or subject, which may be biological or non-biological. As used herein, the term “targeted constituent” includes, but is not limited to, chemical elements, molecules, proteins, analytes, mineral compositions, certain compositions of matter, mineral compositions specific to rocks or ores, DNA, cells, antigens, viruses, and bacteria.

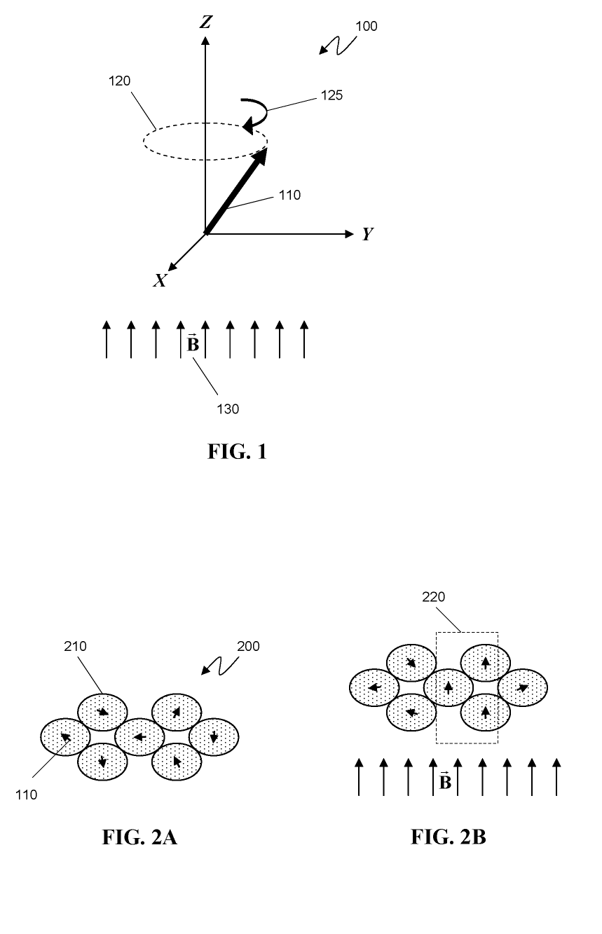

FIG. 1 depicts the dynamics of motion 100 for a single atom's nuclear magnetic moment 110 when placed in an externally-applied static magnetic field \vec{B} 130. Generally, when an atom has a non-zero nuclear spin and is placed in a magnetic field, the atom's magnetic moment 110 precesses in gyroscopic motion about an axis which is substantially aligned with the magnetic field. By way of example as illustrated, the magnetic moment 110 moves about the Z axis, tracing out the path 120 in the direction indicated by arrow 125. The precessional frequency ωₚ depends in part upon the strength of the local magnetic field, i.e., the field in the immediate vicinity of the atom. In various embodiments, the local magnetic field, i.e., the field in the substantially immediate vicinity of the atom, may differ from the applied magnetic field 130 due to material present in the local environment.

A collection of atoms or molecules 210 as depicted in FIG. 2A, e.g., a collection comprising a particle, placed in a substantially uniform and static magnetic field will tend to orient their magnetic moments along the direction of the applied field. This reorientation is referred to as a polarization of the magnetic moments. FIG. 2B illustrates a polarized ensemble of atoms or molecules, e.g., a group of atoms or molecules comprising a particle. The magnetic moments 110 of a fraction of the atoms 220 can reorient in a preferred direction, and the particle takes on a net magnetic moment. When the applied external magnetic field is removed, the orientation of the atoms' moments will randomize at a characteristic rate referred to as the “longitudinal” relaxation time or “spin-lattice” relaxation time T₁. Referring to FIG. 1, during randomization the direction of an atom's magnetic moment 110 will drift in time, away from the path 120, and may point in the −Z direction at a later time. The randomization of all magnetic moments within a collection of atoms can result in zero net magnetic moment for the collection, as depicted in FIG. 2A. In various embodiments, nuclear magnetic resonant signals are derived from the spin-lattice, T₁, relaxation times for a particular species within the particle.

When nuclear magnetic moments for a collection of atoms are polarized and maintained in a substantially static magnetic field, their precessional motion can be substantially synchronized by the application of an RF field tuned to match the precessional frequency ωₚ. The applied field tends to force the precessing moments 110 into synchronous motion. When the applied RF field is removed, the precessing moments begin to drift out of phase with one another. This rate of de-phasing of precessional motion is referred to as the “transverse” relaxation time or “spin-spin” relaxation time T₂. Referring again to FIG. 1, a collection of atoms having their magnetic moments synchronized would exhibit precessional motion 125, 120 in phase with each other.

In various embodiments, NMR signals are derived from the spin-spin, T₂, relaxation properties of a particular species within the particle. In such techniques, sequences of RF fields, tuned to the precession frequency ωₚ for the particular atomic or molecular species, may be applied to the particles. In some embodiments, a short-duration RF field may be applied to synchronize the moments' precessions. After a brief delay, another short-duration RF field may be applied to flip the spin orientation of the nuclear moments. This would correspond to changing the moment's 110 orientation from the +Z direction to the −Z direction in FIG. 1. The spin reversal causes the formerly de-phasing moments to drift back into phase producing a large detectable magnetic impulse or echo when resynchronized. This measurement technique can be repeated many times at a rate slower than about twice the transverse relaxation time, T₂, to improve the signal-to-noise ratio when collecting NMR data.

The strength of the resulting NMR signals and their rates of decay can depend upon several factors including the type of atom or molecule being probed and its local environment. Variations in the local material density and material composition may alter the T₁ time, T₂ time and the precession frequency ωₚ from region to region. These variations can be recorded and plotted to map structural and/or compositional characteristics of the examined sample.

In many applications, NMR signals are derived from the host material itself. For example, in medical imaging the relaxation time, T₁ or T₂ of the hydrogen nucleus (H⁺) is measured. In some applications, NMR signals are derived from naturally occurring atoms, elements, molecules or compounds present within the host material. Although measurements can be made readily in such instances, in certain cases the resulting signals may fail to provide the desired information. For example, NMR remains largely incapable, in present embodiments, to identify chemical biomarkers that may portend malignant cancerous growth or metastasis in a manner suitably efficient and specific to aid in early-stage diagnosis and disease management. Additionally, NMR signals derived from materials or species of atoms native to the host system generally suffer from background or noise NMR signal levels produced by the same species within the host system.

In various embodiments of the inventive methods, NMR-active particles are provided or introduced into a system which is to undergo an NMR measurement. The particles can provide diagnostic telemetry for the system in that the particles provide nuclear-magnetic-resonance signals which can be affected by certain aspects of the system. In some embodiments, the NMR signal provided by the NMR-active particles is located in a spectral region substantially free from any NMR signal originating from the host system, and substantially background-free NMR signals can be detected from the particles. The NMR signals can be derived from the NMR-active particles themselves, e.g., NMR signal intensity and/or frequency location of one or more nuclear magnetic resonance peaks. The signals can be useful for NMR spectroscopic analysis and/or imaging analysis of a system. In certain embodiments, the signals are used to detect the presence of or concentration of a constituent within a system. In some NMR measurements, a shift in the location of a resonance peak is detected. In certain embodiments, the quality of the NMR signals provided by the NMR-active particles is superior to NMR signals derived from material native to the host system, and NMR measurement times can be obtained over short time periods compared to conventional NMR measurement techniques.

The NMR-active particles can be formed from a variety of materials. For example, the particles can be comprised mainly of one or more of the following materials: silicon, silica or carbon. The particles may contain any element, molecule of compound exhibiting an NMR signal when probed with an applied RF excitation field. In some embodiments, the particles may contain a desired element present in a molecule, e.g., fluorine in the form of CaF₂, for which the desired element would provide an NMR signal. In some embodiments, the particles may contain a desired element present as a defect, e.g., nitrogen as a manufactured defect in diamond, for which the desired element would provide an NMR signal. In some embodiments, the NMR-active particles can comprise silicon oxides, which can be coated or partially coated with gold or other metals, for which silicon can provide an NMR signal.

The size of the particles introduced into a system can be distributed over a range of values or distributed about an average value. In some embodiments, particle sizes introduced into a system have a range of values between about 50 nm and about 100 nm, between about 100 nm and 250 nm, between about 250 nm and about 500 nm, between about 500 nm and about one micron, between about one micron and about 5 microns, between about 5 microns and about 20 microns, and yet between about 20 microns and about 100 microns in some embodiments. In some embodiments, the average particle size for a collection of NMR-active particles introduced into a system is any value between about 1 nm and about 200 nm, between about 200 nm and about 1 micron, and yet between about 1 micron and about 200 microns. In some embodiments, the particle size distribution is tens of nanometers, or in some embodiments hundreds of nanometers. In some embodiments, the NMR-active particles have an average particle size d_{avg}, e.g., about 50 nm, about 100 nm, about 150 nm, etc., and the particle size distribution d_{dis} may be expressed as a percentage of the average particle size, e.g., about ±5%, about ±10%, about ±15%, about ±20%, about ±25%, about ±30%, about ±40%, about ±50%, about ±60%, and about ±70%. As an example, NMR-active particles introduced into a system can have an average particle size of about 120 nm, and a particle size distribution of about ±40%. For such a collection of particles, the majority of particles will have a size between about 70 nm and about 170 nm.

Further, the NMR-active particles can have long spin-lattice relaxation times, T₁. In various embodiments, the particles provide NMR signals long after their delivery or introduction into a system. In this context, long periods associated with T₁ relaxation times or long-T₁ times refers to periods longer than about 5 minutes in some embodiments. In various embodiments, the T₁ time is longer than about 15 minutes, longer than about 30 minutes, longer than about one hour, longer than about two hours, and yet in some embodiments longer than about three hours.

There are several techniques that can be used to improve the quality of the NMR signal provided by the NMR-particles. For example, the nuclear magnetic moments of the particles can be polarized through dynamic nuclear polarization either in situ or ex situ. In various embodiments, dynamic nuclear polarization aligns a greater number of the particles' atoms' nuclear magnetic moments in a preferred direction. This can increase the magnitude of NMR signals derived from the particles. Dynamic nuclear polarization can include techniques which utilize any of the following polarization mechanisms: Overhauser effect, solid effect, cross effect and thermal mixing.

In some embodiments, the signal provided by the NMR-active particles is enhanced by isotopic enrichment or depletion of elements within the particles. For example, the particle may be comprised mainly of silicon, with a normal isotopic composition of ²⁸Si (zero nuclear spin, about 92.2% abundant), ²⁹Si (spin=½, about 4.7% abundant) and ³⁰Si (zero spin, about 3.1% abundant). The relative abundance of ²⁹Si may be increased to greater than 5%, greater than 10%, and greater than 20% in some embodiments. In certain embodiments, ²⁹Si can exhibit long T₁ relaxation times, up to several hours. Thus, once the particles are polarized, the increased signal strength can persist for long periods of time. This can be beneficial in embodiments where the particles are injected, ingested, implanted, inhaled or otherwise delivered to living systems and a substantial amount of time is required for the particles to reach an intended destination.

Methods for making NMR-active particles suitable for NMR telemetry as described herein are disclosed in U.S. patent application Ser. No. 12/248,672, filed Oct. 9, 2008, which is incorporated by reference in its entirety.

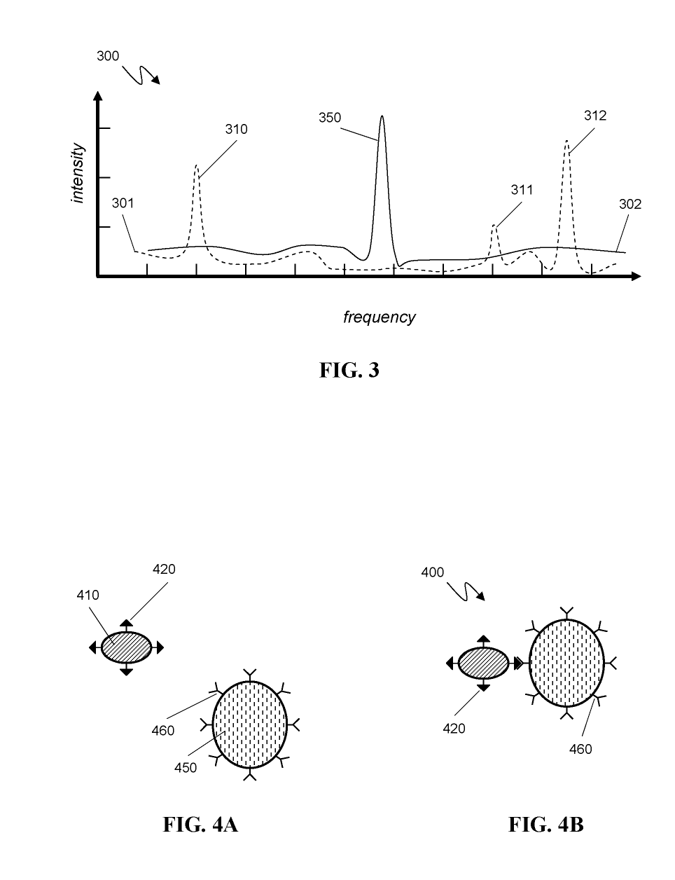

As noted above, the particles can be selected such that they provide an NMR signal in a spectral region that is substantially free from any NMR signals arising from material native to the system. This can result in a high signal-to-noise ratio, and in some embodiments, eliminate a need for spatially-selective probing of a sample. FIG. 3 is a graphical illustration depicting NMR spectra for an embodiment in which the NMR spectrum derived from a NMR-active particle 302, solid curve, has a peak signal 350 located in a spectral region which is substantially free from NMR signals arising from the native material of the system. The native NMR spectrum 301, dashed curve, may exhibit peaks 310, 311, and 312 located in remote regions, but be substantially void of signal in the vicinity of a spectral peak 350 of a selected NMR-active particle. For such embodiments, once the native NMR spectrum is known, an NMR-active particle can be selected for telemetry which exhibits a spectral peak within a substantially signal-free region of the native spectrum. For embodiments having the characteristics depicted in FIG. 3, the signal-to-noise ratio yielded in an NMR measurement of the resonance peak can be greater than about 2, greater than about 5, greater than about 10, greater than about 100 and in some embodiments greater than about 1000. In some embodiments, a resonance peak associated with the NMR-active particles have a signal strength greater than about 2 times the background NMR signal level in the spectral vicinity of the resonance peak. The background signal level can be substantially uniform or may exhibit a peak in the vicinity of the NMR-active particles' resonance peak, and the background signal substantially originates from material native to the system under study. In some embodiments, the NMR-active particles have a signal strength greater than about 5 times the background NMR signal level, greater than about 10 times the background NMR signal level, and yet greater than about 20 times the background NMR signal level.

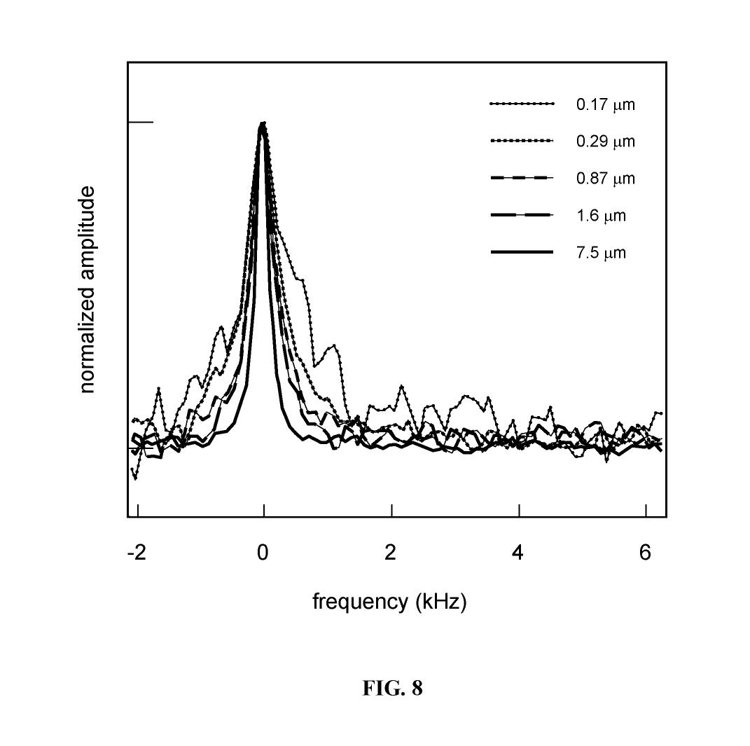

Examples of measured signal strengths as a function of frequency are shown in FIG. 8. The plotted data represents averaged NMR spectra recorded for collections of NMR-active particles of different average sizes. The average particle size for each collection is reported in the graph. The data has been shifted such that the resonance peak is centered about zero frequency value. Each recorded spectra was taken from a series of summed free induction decay traces, following polarization for a time 3T₁ at a magnetic field strength of 4.7 Tesla. The corresponding resonance frequency was about 39.7 MHz. A Bruker DMX-200 NMR console was used for the measurement. The data indicates the quality of the signal can improve with size of the NMR-active particle. In various embodiments, the signal-to-noise ratio of an NMR signal is selectable by selecting an average particle size for any of the inventive methods disclosed herein.

The selection of particles having an NMR signal in a spectral region substantially free from native NMR signals can provide a convenient method of testing for the presence of a targeted constituent within a system without the need for spatially-resolved NMR measurements. As an example, a system potentially containing a targeted constituent, such as cancerous cells, can be exposed to functionalized NMR-active particles having targeting ligands which bind to the cancerous cells or receptors bound to the cancerous cells. If the targeted constituent or receptor-bound constituent is present, the functionalized particles can bind to the targeted constituent or receptor-bound constituent within the system. In some embodiments, after introduction of the functionalized NMR-active particles, the system can be subjected to a cleansing step in which unbound NMR-active particles are removed from the system. A subsequent NMR excitation of the entire system in a narrow range of frequencies encompassing the spectral region around the particle's NMR peak 350 can determine the presence of the particle, and hence the targeted constituent. There would be no need to conduct spatially-resolving, e.g., magnetic resonance imaging, measurements to determine the presence of the targeted constituent for such embodiments.

In various embodiments, the surfaces of the NMR-active particles are chemically altered to provide targeting functionality of the particles. Such chemically-functionalized, NMR-active particles can be used in a variety of applications including, but not limited to, magnetic resonance imaging (MRI), magnetic resonance spectroscopy, and NMR-based agglomeration assays. In some embodiments, the functionalized and targeting NMR-active particles can bind to cell-surface receptors for biological applications, or can bind to rocks or ores having a specific mineral composition for geological applications. In certain embodiments, after binding to a targeted constituent within a system, the functionalized particles can be detected using spatially-selective MRI excitations, and the spatial distribution of targeted constituent within the system, e.g., analytes, cells, types of minerals, etc., can be determined from the resulting NMR signals. As an example, functionalized particles which bind to a localized targeted constituent within a system can provide a “bright” spot on an MRI image, indicating the presence and spatial extent of the targeted constituent. In certain embodiments, after binding to a targeted constituent within a system, the functionalized particles can be detected using non-spatially-selective NMR excitations to determine the presence of a targeted constituent within a system.

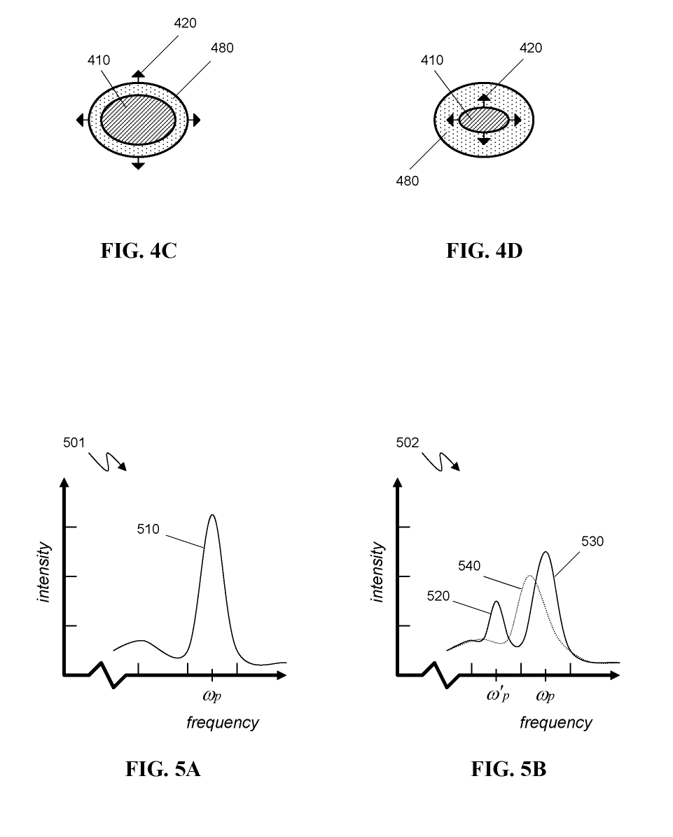

By way of illustration, FIGS. 4A-4B depict an embodiment of a chemically-functionalized, NMR-active particle 410 useful for NMR telemetry. In various embodiments, the surface of an NMR-active particle 410 can be chemically functionalized with targeting ligands 420, as depicted in FIG. 4A. For example, the targeting ligands can comprise any of the following molecules: iodide, bromide, sulfide, thiocyanate, chloride, nitrate, azide, fluoride, hydroxide, oxalate, water, isothiocyanate, acetonitrile, pyridine, ammonia, ethylenediamine, 2,2′-bipyridine, 1,10-phenanthroline, nitrite, triphenylphosphine, cyanide, carbon monoxide, acetylacetonate, various alkenes, benzene, 1,2-bis(diphenylphosphino)ethane, various corroles, various crown ethers, 2,2,2-cryptand, various cryptands, cyclopentadienyl, diethylenetriamine, dimethylglyoximate, ethylenediaminetetraacetate, ethylenediaminetriacetate, glycinate, various hemes, nitrosyl, scorpionate, sulfite, 2,2′,5′,2-terpyridine, thiocyanate, triazacyclononane, tricyclohexylphosphine, triethylenetetramine, tri(o-tolyl)phosphine, tris(2-aminoethyl)amine, tris(2-diphenylphosphinoethyl)amine, terpyridine, polyethylene glycol, dextran, aminopropyltriethoxysilane (APTES), various amines, and various silanes. The targeting ligands may be any of a variety of ligands to which protein molecules will bind. In certain aspects, the targeting ligands may comprise endogenous or exogenous antigens or antibodies. In some embodiments, targeting ligands may comprise ribonucleic acid (RNA). In some embodiments, the targeting ligands are disposed directly on the surface of the particle. In some embodiments, the targeting ligands may be attached to the particle surface through one or more intervening molecules or layers of material.

In various embodiments, the targeting ligands are selected to preferentially bind with a targeted constituent, e.g., a suspected analyte, molecule, protein, biomarker, species or endogenous chemical structures within the system under study. In some embodiments, the targeting ligands bind with cell-surface receptors for biological applications or rocks or ores having specific matter compositions for geological applications. In various embodiments, the functionalized particles are introduced into a system and detected directly using spatially-selective, magnetic-resonance-imaging (MRI) excitations. Spatially-selective MRI excitations can include spatially-varying static magnetic fields, e.g., fields having intensity gradients along at least one dimension of space, as would be known to one of ordinary skill in the art of magnetic resonance imaging. The spatial distribution of the targeted constituents can be determined from an imaged constructed from recorded NMR signals derived from the functionalized NMR-active particles. As an example, an accumulation of functionalized particles at a particular location within a system can be representative of multiple binding events between targeted constituents and functionalized particles, and this accumulation can be evident as a localized increase in NMR signal strength, e.g., a bright spot on an MRI image.

In some embodiments, the particle may comprise an NMR-active core encased or encapsulated in a polymer shell. The polymer shell may be bioabsorbable or biodegradable. Exemplary biodegradable materials include any of the following polymers: lactide-glycolide copolymers of any ratio (e.g., 85:15, 40:60, 30:70, 25:75, or 20:80), polyesters, polycarbonates, polyamides, polyethyleneglycol, and polycaprolactone. For embodiments as depicted in FIG. 4C, where a biodegradable polymer shell 480 encapsulates an NMR-active core 410, the targeting ligands 420 may be disposed on the outer surface of the shell or on the surface of the active core as depicted in FIG. 4D. The embodiment corresponding to FIG. 4D can provide time-delayed targeted delivery of the NMR-active particles. In some embodiments, therapeutic drugs can be incorporated into the shell 480. In embodiments as depicted in FIG. 4C, wherein therapeutic drugs are disposed within shell 480, delivery of drugs to a targeted receptor, e.g., a receptor 460 which preferentially binds with targeting ligand 420, can be tracked within a system.

Referring again to FIG. 4A in certain embodiments, a chemically-functionalized, NMR-active particle 410 can be introduced into a system in which a binding site for the targeting ligand is believed to be or suspected to be present. The binding site, or receptor 460, may be disposed on the surface of a targeted constituent, e.g., a complex molecule, cell or structure 450 within the system, may be contained within a targeted constituent, or may be unattached and freely moving within the system. As an example, the receptor 460 may be human antigens disposed on the surface of red blood cells, and the targeting ligand on the NMR-active particle may be a human antibody which targets the antigen. As an additional example, the binding site may be a particular chemical element, molecule, or protein not normally present to the system, and the targeting ligand can bind to that particular element, molecule or protein. As additional examples, the targeted constituents can be receptors on islet cells within the pancreas, or receptors on cancerous cells or malignancies within any biological organ, e.g., any human organ such as the prostrate, kidney, liver, lungs, etc. or any animal organ. In various embodiments, a chemically-functionalized, NMR-active particle will bind to the targeted receptor 460 through the targeting ligand 420 as depicted in FIG. 4B. When the particle 410 has more than one targeting ligand on its surface, additional binding can occur and form an agglomeration of particles and receptors or receptor-bound targeted constituents.

In some embodiments, a shift in the resonance peak of the NMR-active particles can occur after introduction of the particles into a system. The shift in resonance peak can be detected by carrying out an NMR measurement on the system in a spectral region encompassing the resonance peak and its vicinity. In some embodiments, the NMR measurement to detect the shift or change in the resonance peak requires a brief period of time, e.g., between about 10 minutes and about 20 minutes, between about 5 minutes and about 10 minutes, between about 2.5 minutes and about 5 minutes, between about 1 minutes and about 2.5 minutes. In some embodiments, the data acquisition time for the NMR measurement is between about 10 seconds and about 1 minute. In some embodiments, a shift in the resonance peak is representative of a concentration of a targeted constituent within the system.

By way of example, when a functionalized, NMR-active particle binds with a targeted constituent such as a receptor or receptor-bound particle, a change in the NMR-active particle's spectral characteristics can result. Such a change is depicted in FIGS. 5A-5B. For example, an unbound NMR-active particle 410 as depicted in FIG. 4A may exhibit an NMR spectrum 501 as depicted in FIG. 5A. The NMR spectrum can be obtained by sweeping the frequency of the applied RF excitation fields and recording the resulting NMR signal strength. The NMR spectrum may exhibit a dominant resonance peak 510 at a frequency ωₚ corresponding to a nuclear-magnetic-active species present in the particle 410.

After binding 400 with a targeted constituent, the NMR spectrum can become altered, as depicted in the illustrated example of FIG. 5B. The bound spectrum 502 can exhibit a new satellite peak 520 at frequency ω′ₚ and a reduced main peak 530, as indicated by the solid curve. The satellite peak can result from the bound particles 400 in the system wherein the bound targeted constituent affects the local magnetic field for the particle and therefore alters its magnetic resonance frequency. The remaining unbound particles 410 still contribute to the main peak 530. In some embodiments, the shift in frequency of the bound particles may be too small to resolve by instrumentation as a separate spectral peak, and a broadened, shifted peak 540 may result as depicted by the dotted curve in FIG. 5B.

It will be appreciated that the illustrated spectrum of FIG. 5B is only one example of how the NMR spectrum can be altered. In some embodiments, the resulting spectrum may exhibit only a satellite peak 520, e.g., if substantially all NMR-active particles become bound or if non-bound particles are cleansed from the system. In some embodiments, the resulting spectrum may exhibit a broadened main peak, or double-peaked resonance structure.

In certain embodiments, the intensity, shape, and/or location of the spectral peaks 520, 530, or 540 can provide quantitative information about the extent and/or concentration of binding of the particles to targeted constituents. For example, in some systems extensive and concentrated binding of functionalized particles to targeted constituents can produce larger shifts in the NMR resonant frequency than moderate binding, or can produce a measurable increase in signal strength, e.g., intensity or peak value. In some embodiments, a shift or change in a magnetic resonance peak can be pre-calibrated, and the signal intensity of peaks at characteristic shifts can give quantitative information about targeted constituent, e.g., concentration of the constituent present in the system. Pre-calibration trials can be carried out to measure shifts or change in the particles' resonance peak as a function of known concentration of the targeted constituent.

In some embodiments, the binding of a functionalized NMR-active particle to a targeted constituent can affect the particle's T₁ and/or T₂ times. These changes can be detected via an NMR measurement to determine the presence of the targeted constituent. In some magnetic-resonance imaging embodiments, plural aspects or characteristics of signals provided by the NMR-active particles are detected or measured to provide additional information. For example, any combination or all of the following aspects and/or their changes can be detected in an NMR imaging measurement: signal intensity, signal frequency, spectral characteristics of a resonance peak, T₁ time, and T₂ time. A resulting image can be weighted by or associated with any of the one or more measured aspects and/or their changes. As one example, an image based on signal intensity can be accompanied with an image based on changes in T₂ time. As an additional example, spatial imaging weighted by resonance frequency data can provide a spatial-spectral image of the system.

It will be appreciated that NMR-active particles providing signals in a frequency band substantially free of background or noise signals can yield a high signal-to-noise ratio in any of the aforementioned NMR measurements. In various embodiments, any type of NMR measurement carried out with the inventive NMR-active particles can acquire data in time periods less than those required for conventional NMR measurement techniques. In various embodiments, a measurement to detect an NMR signal for any of the aforementioned aspects and/or their changes can require a time period between about 10 minutes and about 20 minutes, between about 5 minutes and about 10 minutes, between about 2.5 minutes and about 5 minutes, between about 1 minutes and about 2.5 minutes. In some embodiments, the data acquisition time for the NMR measurement is between about 10 seconds and about 1 minute.

By way of further example, chemically-functionalized, NMR-active particles can be used in combination with functionalized paramagnetic or superparamagnetic particles, e.g., iron oxide particles, gadolinium particles or particles with similar properties, in a multi-particle agglomeration assay adapted for NMR telemetry. In certain embodiments, the inventive methods are employed in agglomeration assays where the assay contains NMR-active particles, which can be chemically functionalized, and superparamagnetic or paramagnetic particles. In some embodiments, the paramagnetic or superparamagnetic particles are iron oxide or gadolinium, and their surfaces can also be functionalized. The NMR-active particles can be introduced into an assay system containing superparamagnetic or paramagnetic particles. In various embodiments, the further addition of an analyte causes agglomeration of the NMR-active particle, the analyte, and the superparamagnetic or paramagnetic particles. Agglomeration can result in a net shift of the nuclear magnetic resonance peak of the NMR-active particle. For example, a concentration of superparamagnetic or paramagnetic particles in the vicinity of the NMR-active particles due to agglomeration can alter the local magnetic field for the NMR-active particles and affect any or all of the following aspects or characteristics of the particles: resonance frequency, T₁ time, T₂ time. In certain embodiments, a shift in resonance frequency can be detected and can provide quantitative information about analyte concentration.

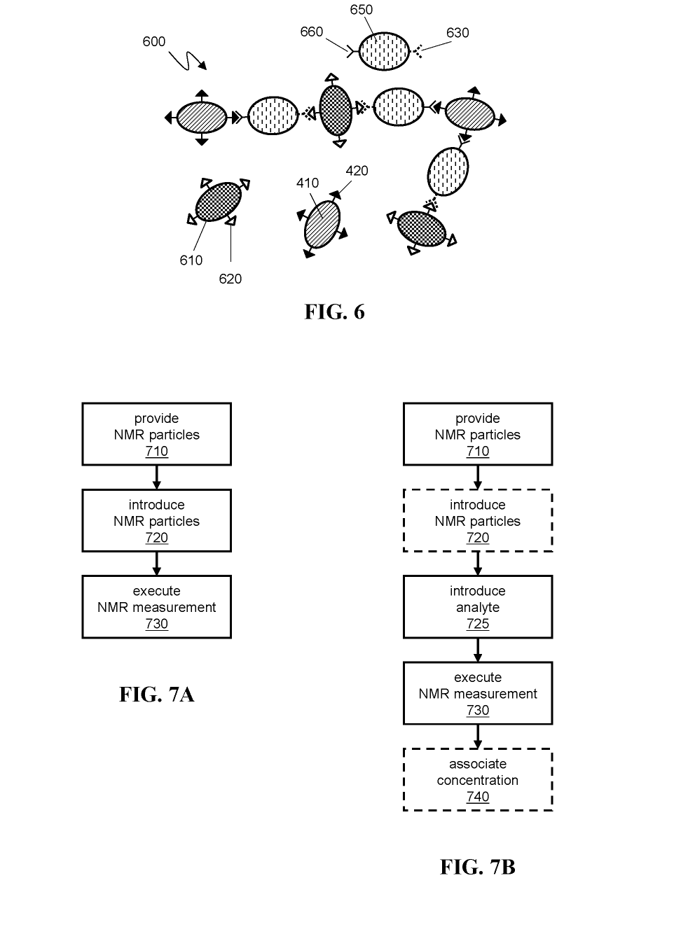

By way of further example, an embodiment of an assay employing multi-particle agglomeration including paramagnetic or superparamagnetic particles and NMR-active particles is depicted in FIG. 6. In the illustrated embodiment, a targeted constituent 650 has two functionally different receptors 630 and 660. A targeting ligand 420 may be disposed on the surface of an NMR-active particle 410, and ligand 420 may preferentially bind with receptor 660. A second targeting ligand 620 may be disposed on the surface of paramagnetic particle 610, and its targeting ligand 620 may preferentially bind with receptor 630. The size of the paramagnetic or superparamagnetic particles can be less than about 50 nanometers (nm), between about 50 nm and about 100 nm in some embodiments, between about 100 nm and 250 nm, between about 250 nm and about 500 nm, and between about 500 nm and one micron in some embodiments. As the agglomeration 600 forms, the paramagnetic particles 610 can become bound in the matrix in close proximity to the NMR-active particles 410, and locally alter any applied magnetic field. The agglomeration can cause a shift in the NMR resonance peak associated with the NMR-active particles 410. In some embodiments, the amount of the shift, change in shape, and/or the intensity of the NMR signal can provide quantitative information about the amount and/or concentration of the targeted constituent, e.g., an analyte, present in the assay.

In some embodiments, both functionalized NMR-active particles and paramagnetic or superparamagnetic particles are introduced into a system, e.g., into a human or animal subject or biological sample. The NMR-active particles and magnetic particles can be similarly functionalized to target a specific constituent within the system, e.g., cancerous growth. Accumulation of the NMR-active particles and magnetic particles at a localized site can result in a spectral shift of the NMR-active particles' resonance peak and indicate the presence of a cancerous growth within the system.

In certain embodiments, the accumulation of NMR-active particles within a system can be detected using spatially resolving measurement techniques such as magnetic-resonance imaging (MRI). Imaging in this context is understood to be NMR detection where the spectroscopic intensity of signals derived from the particles can be mapped to spatial locations within the system to form an MRI image of at least a portion of the system. In some embodiments, shifts in the resonance peak of the NMR-active particles can be mapped to spatial locations to form an MRI image. Imaging techniques can include the use of one or more magnetic field gradients. The intensity of the image in various regions yields information about relative concentration of the particles or of targeted constituents within certain regions of the system and, in some embodiments can even be used to quantify absolute concentrations of particles or constituents within the regions. The quantification of concentrations can be obtained by comparing measured results with results from pre-calibration trials.

In various embodiments, spatial resolution exceeding values obtained by conventional MRI techniques are obtained with the inventive NMR telemetry methods. In certain embodiments, the spatial resolution obtained for imaging is between about 5 milliliters and about 10 milliliters, between about 2.5 milliliters and about 5 milliliters, and yet in some embodiments between about 1 milliliters and about 1.5 milliliters. Images constructed from signals derived from the NMR-active particles can be two-dimensional or three-dimensional representations of at least a portion of the system into which the NMR-active particles are introduced.

In some embodiments, image intensity derived from functionalized particles in MRI applications can provide information useful for analysis, diagnosis and/or treatment of a system. By way of example, the kinetics of particles in vivo, in vitro or in situ can be affected by certain parameters, e.g., specific gravity, size and surface composition of the particle. When two of these parameters are held to be constant, e.g., by using particles of a selected uniform size and specific gravity, variations in kinetics, e.g., physiological distribution, rate of decomposition, etc, can provide information about characteristic interactions within a system relating to the third parameter, surface chemistry in this example. Many biological processes are mediated through contact interactions between extracellular biomolecules and cellular surface receptors, and these interactions can trigger a number of processes broadly referred to as “cellular functions.” Cellular functions can include, for example, but not be limited to, changes in gene expression, changes in the cell lifecycle, and adaptive responses to extracellular stimuli. In various instances, the types of surface receptors present on the surface of a cell can be characteristic of a class of cells, e.g., insulin producing islet cells, malignant cancer cells, or cells of the immune system, and can be indicative of an adaptive response to an extracellular stimulus. In various embodiments, accumulation of functionalized particles in various regions of a system, where the accumulation is caused by modified kinetic properties of the particles in those regions due to interactions of the targeting ligands with targeted receptors, or larger physiological structures including vasculature, is indicative of the presence of those receptors, and can provide useful information about cell type and cellular function. Accumulation of functionalized particles can become evident as an increase in NMR signal strength during magnetic resonance imaging (MRI). In some embodiments, detected accumulations of functionalized particles can, for example, provide information to physicians about types of cells present in different tissues or, in the case of chemotherapeutics, about whether administered drugs are deposited in the tissues for which they are targeted.

Various embodiments of methods for NMR telemetry using NMR-active particles are depicted in the flow charts of FIGS. 7A-7B. These methods can include the use of chemically-functionalized, NMR-active particles, and chemically-functionalized paramagnetic or superparamagnetic particles. The methods can include the use of NMR-active particles with long T₁ relaxation times.

In various embodiments, NMR-active particles are selected, obtained or provided 710. In some embodiments, the selected NMR-active particles have been chemically functionalized. In some embodiments, the NMR-active particles have an NMR resonance peak in a spectral region which is substantially free from an NMR signal originating from a system into which the particles will be introduced. The step of providing the NMR-active particles can include polarizing at least a portion of the nuclear magnetic moments of the particles.

A method of telemetry for nuclear magnetic resonance can further include introducing 720 the NMR-active particles into the system. The step of introducing can include introducing the particles, or a solution containing the particles, into the system. The system can contain a constituent known or suspected to be present within the system. In some embodiments, the system is an agglomeration assay. The particles can be introduced to the system in solution, as a powder, as a dissolvable tablet, or as an encapsulated composition. The particles, in various forms, can be introduced by infusion, injection, ingestion, inhalation, intravenous delivery, per os, per anus, transdermal delivery, etc or implantation. In certain embodiments, a selected period of time is allowed to elapse after introduction of the NMR-active particles into the system. The selected period of time can provide for dispersion of the NMR-active particles throughout the system. In some embodiments, mixing techniques are employed to accelerate dispersion of the NMR-active particles throughout the system. In certain embodiments, the selected period provides time for the particles to reach a targeted destination. The intermixing or dispersion of particles can be accomplished in a variety of manners including mechanical stirring, shaking, tumbling, ultrasonic agitation, or natural diffusion and dispersion of the particles, as well as the targeted constituents in some cases. In various embodiments, the intermixing is provided for a pre-selected amount of time lasting between about 30 seconds and one minute, between about one minute and about 10 minutes, between about 10 minutes and about 30 minutes, and between about 30 minutes and about one hour. The step of introducing 720 can further comprise enhancing the NMR signal provided by the particles using techniques of dynamic nuclear polarization, which may be carried out in situ or ex situ. The dynamic nuclear polarization acts to polarize the nuclear magnetic moments of atoms within the particles.

A method of telemetry for nuclear magnetic resonance can further include executing 730 an NMR measurement on the system. The measurement can be spatially-resolving in some embodiments, and non-spatially-resolving in additional embodiments. In some embodiments, the NMR measurement detects a shift in the resonance peak of the NMR-active particles. In some embodiments, the NMR measurement detects an intensity value of the resonance peak of the NMR-active particles. In some embodiments, the NMR measurement detects both an intensity value of and a shift in the resonance peak. As noted above, the spatial resolution obtainable for imaging measurements can exceed values obtained by conventional magnetic resonance imaging techniques. In some embodiments, the time required for executing 730 an NMR measurement, e.g., the time required to acquire data representative of resonance signals from the NMR-active particles, can be less than times required for conventional NMR measurement techniques. In some embodiments, the NMR measurement requires between about 10 minutes and about 20 minutes, between about 5 minutes and about 10 minutes, between about 2.5 minutes and about 5 minutes, and yet in some implementations between about 1 minute and about 2.5 minutes.

FIG. 7B depicts an embodiment of a method of telemetry for nuclear magnetic resonance which further comprises additional steps of introducing 725 an analyte into the system and associating 740 a concentration with a result of the NMR measurement, e.g., shift in resonance peak. The method depicted in FIG. 7B can be utilized in NMR agglomeration assays. In certain embodiments, the step of introducing the NMR-active particles is omitted. For example, the NMR-active particles can be provided in an agglomeration assay system, such as a test tube, vial, small dish or well, microtitre place, multi-well assay plate, etc. The step of associating 740 can be optional and can take various forms. For example, in certain embodiments, the step of associating 740 can comprise determining an approximate value for the concentration of analyte introduced into the system. The determination can be made from data previously acquired during pre-calibration trials. In certain embodiments, the step of associating 740 can comprise a threshold-determination procedure. For example, a detected shift in a resonance peak or change in intensity level beyond a threshold value provides a positive, or in some embodiments a negative, indication of the presence of a targeted constituent.

All literature and similar material cited in this application, including, but not limited to, patents, patent applications, articles, books, treatises, and web pages, regardless of the format of such literature and similar materials, are expressly incorporated by reference in their entirety. In the event that one or more of the incorporated literature and similar materials differs from or contradicts this application, including but not limited to defined terms, term usage, described techniques, or the like, this application controls.

The section headings used herein are for organizational purposes only and are not to be construed as limiting the subject matter described in any way.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art.

The claims should not be read as limited to the described order or elements unless stated to that effect. It should be understood that various changes in form and detail may be made by one of ordinary skill in the art without departing from the spirit and scope of the appended claims. All embodiments that come within the spirit and scope of the following claims and equivalents thereto are claimed.

What is claimed is:

1. A method for remotely determining whether a system exhibits a characteristic by nuclear magnetic resonance comprising:
providing NMR-active particles having an NMR resonance peak;
introducing the NMR-active particles into a system;
detecting the resonance peak of the NMR-active particles with NMR apparatus;
determining whether the resonance peak of the NMR-active particles shifts as a result of being introduced into the system; and
determining that the system exhibits or does not exhibit a characteristic based on the occurrence or non-occurrence of a shift.

2. The method of claim 1, wherein the NMR resonance peak of the NMR-active particles is in a spectral region which is substantially free from any NMR signal originating from the system.

3. The method of claim 1, wherein the resonance peak of the NMR-active particles splits into two or more resonance peaks as a result of being introduced into the system.

4. The method of claim 1, wherein the resonance peak of the NMR-active particles broadens as a result of being introduced into the system.

5. The method of claim 1, wherein the NMR-active particles bind a characteristic analyte within the system and the NMR resonance peak of the NMR-active particles shifts when bound to the analyte.

6. The method of claim 1, wherein the system is an organism and the analyte is a characteristic cell type.

7. The method of claim 6, wherein the analyte is a characteristic cancer cell type.

8. The method of claim 1, wherein the NMR-active particles are chemically functionalized.

9. The method of claim 1, wherein the NMR-active particles have undergone isotopic enrichment or isotopic depletion.

10. The method of claim 1 further comprising enhancing a nuclear magnetic resonance signal originating from the NMR-active particles by dynamic nuclear polarization, the dynamic nuclear polarization performed in situ or ex situ.

11. The method of claim 1, wherein the resonance peak has a signal strength greater than about 2 times the background NMR signal level.

12. The method of claim 1, wherein the resonance peak has a signal strength greater than about 5 times the background NMR signal level.

13. The method of claim 1, wherein the resonance peak has a signal strength greater than about 10 times the background NMR signal level.

14. The method of claim 1, wherein the resonance peak has a signal strength greater than about 20 times the background NMR signal level.

15. The method of claim 1, wherein the detection of the shift in resonance peak is done using spatially resolving measurement techniques.

16. The method of claim 15, wherein the spatial resolution is between about 5 milliliters and about 10 milliliters.

17. The method of claim 15, wherein the spatial resolution is between about 2.5 milliliters and about 5 milliliters.

18. The method of claim 15, wherein the spatial resolution is between about 1 milliliter and about 2.5 milliliters.

19. The method of claim 1, wherein the detection of the shift in resonance peak is done without using spatially resolving measurement techniques.

20. The method of claim 1, wherein a measurement to detect the shift in resonance peak requires between about 10 minutes and about 20 minutes.

21. The method of claim 1, wherein a measurement to detect the shift in resonance peak requires between about 5 minutes and about 10 minutes.

22. The method of claim 1, wherein a measurement to detect the shift in resonance peak requires between about 2.5 minutes and about 5 minutes.

23. The method of claim 1, wherein a measurement to detect the shift in resonance peak requires between about 1 minute and about 2.5 minutes.

24. The method of claim 1 further comprising associating a concentration with the detected shift in resonance peak.

25. A method of telemetry for nuclear magnetic resonance assays comprising:
providing NMR-active particles having an NMR resonance peak in a spectral region which is substantially free from any NMR signal originating from other components in an assay system;
introducing the NMR-active particles into the assay system;
introducing an analyte into the assay system; and
detecting a shift in the resonance peak of the NMR-active particles.

26. The method of claim 25 further comprising associating a concentration of the analyte with the detected shift in resonance peak.

27. The method of claim 25, wherein the NMR-active particles are chemically functionalized.

28. The method of claim 25, wherein the NMR-active particles have undergone isotopic enrichment or isotopic depletion.

29. The method of claim 25 further comprising enhancing a nuclear magnetic resonance signal originating from the NMR-active particles by dynamic nuclear polarization, the dynamic nuclear polarization performed in situ or ex situ.

30. The method of claim 25, wherein the resonance peak has a signal strength greater than about 2 times the background NMR signal level.

31. The method of claim 25, wherein the resonance peak has a signal strength greater than about 5 times the background NMR signal level.

32. The method of claim 25, wherein the resonance peak has a signal strength greater than about 10 times the background NMR signal level.

33. The method of claim 25, wherein the resonance peak has a signal strength greater than about 20 times the background NMR signal level.

34. The method of claim 25, wherein the detection of the shift in resonance peak is done without using spatially resolving measurement techniques.

35. The method of claim 25, wherein a measurement to detect the shift in resonance peak requires between about 10 minutes and about 20 minutes.

36. The method of claim 25, wherein a measurement to detect the shift in resonance peak requires between about 5 minutes and about 10 minutes.

37. The method of claim 25, wherein a measurement to detect the shift in resonance peak requires between about 2.5 minutes and about 5 minutes.

38. The method of claim 25, wherein a measurement to detect the shift in resonance peak requires between about 1 minute and about 2.5 minutes.
